(19) United States
(12) Patent Application Publication
(10) Pub. No.: US 2017/0130439 A1
(43) Pub. Date: May 11, 2017

(54) DEODORIZING SYSTEM, DEVICE AND METHODS FOR ODOR REMOVAL

(71) Applicants: R VALLEY FARMING CO. LTD., Assiniboia (CA); GRANTA MANUFACTURING INC., Bassano (CA)

(72) Inventors: JOHN S. KLEINSASSER, Assiniboia (CA); MIKE P. WIPF, Bassano (CA)

(21) Appl. No.: 15/114,299

(22) PCT Filed: Jan. 23, 2015
(86) PCT No.: PCT/CA2015/050045

§ 371 Date: Jul. 26, 2016

Related U.S. Application Data
(60) Provisional application No. 61/931,965, filed on Jan. 27, 2014.

(51) Int. Cl. E03D 9/052; A61L 9/014; A61L 9/16; B01D 46/00; B01D 46/44

(52) U.S. Cl. CPC E03D 9/052; B01D 46/0038; B01D 46/44; B01D 46/446; B01D 46/0086; A61L 9/16; A61L 9/014; A61L 2209/111; A61L 2209/14

(57) ABSTRACT

A method for deodorizing air at or near a toilet is provided. The method comprises detecting pressure on the toilet seat; directing ah from near or inside the toilet bowl through a filter at a first rate if pressure on the toilet seat is detected; detecting presence of H₂S; directing air from near or inside the toilet bowl through the filter at a second rate if H₂S is detected, the second rate being higher than the first rate; detecting removal of pressure from the toilet seat; and ceasing the direction of air through the filter after a predetermined period of time if no H₂S is detected or if removal of pressure on the toilet seat is detected. A system and a device relating to the method are also provided.

### FIELD

The present invention generally relates to a deodorizing system, and in particularly exemplary embodiments to a deodorizing system useful for toilet odor removal. The present invention also relates to a deodorizing device and methods of odor removal.

### BACKGROUND

Numerous devices have been proposed for the removal or neutralization of objectionable, obnoxious odors generated from defecation in toilets. While such devices are generally intended for improving sanitation and air quality by permitting the withdrawal of malodorous fumes for treatment or for expulsion to an outside environment, the devices suffer from drawbacks. For example, known ventilation devices are cumbersome, and typically contain unsightly vent hoses or pipes that make them aesthetically unacceptable to discerning consumers. Further, installation of ventilation devices frequently requires expensive and permanent modifications to the bathroom structural interiors and/or to the toilet itself.

Conventional filtration and deodorizing devices are designed to eliminate odors and then exhaust purified air back into the bathroom. Such devices negate the need for extensive installation procedures, lengthy vent hoses or pipes, and costly toilet and room modifications. Systems that recirculate air through a filter in multiple passes may be more effective in neutralizing odors, but do so at the cost of higher energy consumption, which can be particularly problematic when the device is powered by battery which has a finite energy capacity. The need to routinely replace the battery in these filtration systems is inconvenient and costly for the consumer.

Other devices add fragrance to processed air to mask odors. The scents of such fragrances are not always effective in their masking, and are not acceptable to many consumers. As a consequence, conventional deodorizing devices have not been broadly accepted and have enjoyed limited commercial success.

### SUMMARY OF INVENTION

According to a broad aspect of the present invention, there is provided a method for deodorizing air at or near a toilet comprising a toilet seat, a toilet tank, and a toilet bowl, the method comprises: detecting pressure on the toilet seat; directing air from near or inside the toilet bowl through a filter at a first rate if pressure on the toilet seat is detected; detecting presence of H₂S; directing air from near or inside the toilet bowl through the filter at a second rate if H₂S is detected, the second rate being higher than the first rate; detecting removal of pressure from the toilet seat; and ceasing the direction of air through the filter after a predetermined period of time if no H₂S is detected or if removal of pressure on the toilet seat is detected.

According to another broad aspect of the present invention, there is provided a deodorizing system for operation with a toilet having a toilet seat and a water tank, the system comprising: a controller; a pressure sensor for sensing pressure on the toilet seat and for communicating same to the controller via a first signal; an air filtration system for filtering air near the toilet upon receiving the first signal from the controller; and a power source for supplying power to the controller and the air filtration system.

According to yet another broad aspect of the present invention, there is provided a device for deodorizing air at or near a toilet comprising a toilet seat, a toilet tank, and a toilet bowl, the device comprises: a outer housing having a perforated first end and a perforated second end; a filter medium disposed inside and between the first and second ends of the outer housing; a suction device disposed inside the outer housing, the suction device configured to draw air through the filter medium when in operation; an odor sensor disposed on or inside the outer housing, the odor sensor being in communication with the controller; a pressure sensor in communication with the controller, the pressure sensor for detecting changes in pressure on the toilet seat; a controller disposed inside the outer housing for receiving signals from the pressure sensor and the odor sensor, and for selectively operating the suction device; a power source electrically connected to the suction device, the controller, the odor sensor and the pressure sensor, for supplying power to same; and a conduit assembly connected to the first end of the outer housing, for facilitating the suction of air from near or inside the toilet by the suction device and/or for directing the air through the odor sensor and the filter medium.

### BRIEF DESCRIPTION OF THE DRAWINGS

Drawings are included for the purpose of illustrating certain aspects of the invention. Such drawings and the description thereof are intended to facilitate understanding and should not be considered limiting of the invention. Drawings are included, in which:

FIG. 1 is a schematic diagram of one embodiment of the present invention;

FIG. 2 is a schematic diagram of another embodiment of the present invention, showing various optional components;

FIG. 3 is a flowchart of a sample method according to one embodiment of the present invention;

FIG. 4 is a flowchart of a sample method according to another embodiment of the present invention;

FIG. 5 is a flowchart of a sample method according to yet another embodiment of the present invention;

FIG. 6a is a side view of a device according to an exemplary embodiment of the present invention;

FIG. 6b is a perspective view of the device in FIG. 6a;

FIG. 6c is another perspective view of the device in FIG. 6a;

FIG. 7 is a cross-sectional view of the device along the line A-A in FIG. 6a; and

FIG. 8 is an exploded view of the device in FIG. 6a.

### DETAILED DESCRIPTION OF VARIOUS EMBODIMENTS

The detailed description set forth below in connection with the appended drawings is intended as a description of exemplary embodiments of the present invention and is not intended to represent the only embodiments contemplated by the inventor. The detailed description includes specific details for the purpose of providing a comprehensive understanding of the present invention. However, it will be apparent to those skilled in the art that the present invention may be practiced without these specific details.

The present invention operates to sense, collect and filter malodorous air resulting from, for example, defecation in toilets. Particularly, the invention aims to sense and remove hydrogen sulfide (H₂S), a foul smelling gas emitted during defecation, from the surrounding air. In a preferred embodiment, the deodorizing system is designed to be self-powered. In a further preferred embodiment, the device of the present invention is configured to be mountable on the toilet without any disassembly, substitution of replacement parts, or permanent modification of the toilet or its components or existing plumbing. The device is installable on a wide variety of toilets, including those not equipped with a toilet lid. The device may also be used with “Porta-Potties,” which are mobile toilet sheds, marine heads, one-piece molded toilets, etc. The device may help in reducing unnecessary water usage in the toilet.

Although illustrated and described primarily with respect to the removal of odors from a toilet or bathroom area, it should be understood that the present invention in its broader aspects has universal deodorizing applications. Alternative applications may include elimination or reduction of cigarette smoke from residential or office environments, reduction or removal of solvents and lacquer vapor odors from nail technician locations, RV compartment deodorizers, or general open area or closed room odor removal.

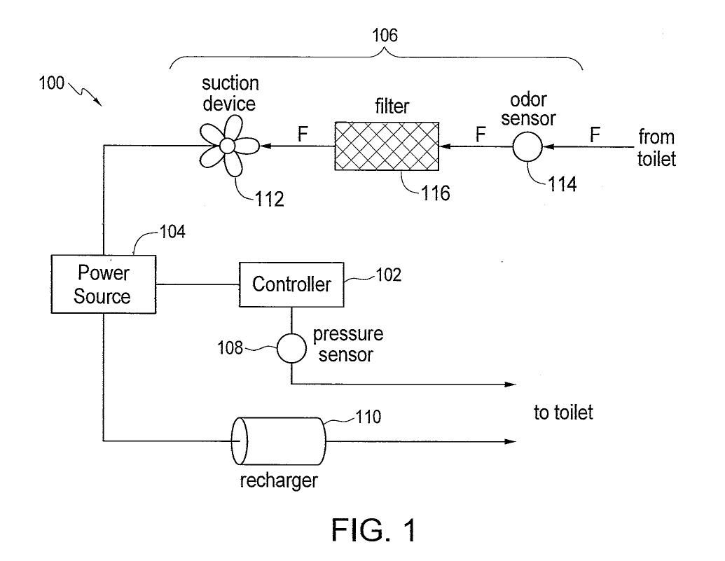

With reference to FIG. 1, a deodorizing system 100 for operation with a standard toilet (not shown) according to one embodiment of the present invention comprises a controller 102, a pressure sensor 108, a power source 104, a recharger 110, and an air filtration system 106. Controller 102 communicates and controls various components of the system. In the illustrated embodiment, controller 102 is in communication with power source 104 and a pressure sensor 108, for sending and/or receiving electronic signals thereto and/or therefrom. In one embodiment, controller 102 is a microprocessor (“μP”).

Power source 104 supplies power to one or more components in the system. In the illustrated embodiment, the power source 104 is in communication with filtration system 106 and controller 102 for supplying power thereto, as necessary, and also in communication with the recharger 110 for receiving power therefrom, as described in more detail hereinbelow. Preferably, power source 104 supplies DC power and is capable of recharging itself by receiving power from another source. Power source 104 may comprise and/or use a voltage regulating device to control its output voltage levels. The voltage regulating device includes for example a voltage regulating semiconductor, such as Zener diodes.

In a sample embodiment, power source 104 comprises ultra-capacitors (sometimes also referred to as supercapacitors or electric double-layer capacitors (EDLC)). In general, ultra-capacitors have the highest available capacitance values per unit volume and the greatest energy density of all capacitors. They support up to 12,000 farads/1.2 volt, with capacitance values up to 10,000 times that of electrolytic capacitors. Ultra-capacitors generally have power densities that are about 10 to 100 times greater than that of a conventional battery. Power density is defined as the product of energy density multiplied by the speed at which the energy is delivered to the load. The greater power density results in much higher charge and discharge speeds than those of a battery, and a greater tolerance for numerous charge and discharge cycles than that of a battery. In a further sample embodiment, power source 104 comprises four ultra-capacitors that are connected in series to provide approximately 10.8 V. In one embodiment, the ultra-capacitor's voltage levels are controlled, for example, using Zener diodes to help extend the life of the ultra-capacitors and avoid critical failure of same. Of course, any other device that is capable of repeated charging and discharging cycles and can supply DC power may be used for power source 104.

The recharger 110 is any device that can convert hydraulic power to electrical power. For example, in one embodiment, recharger 110 is connectible to the toilet and installable somewhere in the flow path of the water intake and/or inlet of the toilet's water tank, such that each time the water tank is refilled, the flow of water coming into the tank drives the recharger. As water coming into the tank drives the recharger, the recharger generates electrical power (e.g. AC voltage and current) and supplies same to the power source, to recharge the power source.

A device that may be used for recharger 110 is a micro-turbine. The micro-turbine is a small-scale water turbine for powering an electrical generator. In general, micro-turbines have an ability to be turned on and off within a short period of time (i.e. seconds or minutes), supplying power almost instantaneously. In a sample embodiment, the micro-turbine is installed in line with the toilet tank water supply inlet. Water moving through the turbine drives an alternator to produce electricity for the power source. Of course, other devices that can convert hydraulic power to electrical power may be used.

The recharger 110 is preferably is selected to generate sufficient power to substantially fully recharge the power source 104 each time the toilet tank is filled. In sample embodiment, the recharger provides approximately 12 VDC at around 48 mA to recharge the power source 104. In a further sample embodiment, the recharger produces 28 VAC p-p with 200 mA at 1.2 Khz with a 150 Ohm load at full water flow during tank refill (e.g. approximately 6 liters per minute).

If necessary, recharger 110 may comprise and/or use addition components, such as for example a bridge rectifier and/or electrolytic capacitor, to convert the power it generates into a form that is receivable and usable by power source 104. Alternatively or additionally, power source 104 may comprise and/or use addition components, such as for example a bridge rectifier and/or electrolytic capacitor, to convert the power it receives from recharger 110 into a form that it can use to recharge itself.

Recharger 110 and/or power source 104 may further include and/or use an additional component(s) configured to ensure that power source 104 is not recharged past its maximum voltage.

Pressure sensor 108 is connectible to the toilet and installable at a location on the toilet where the sensor 108 can sense weight and/or pressure on the toilet seat. The purpose of pressure sensor 108 is to detect and measure pressure, acceleration, strain and/or force and converting same into an electric signal. Various types of sensors may be used for pressure sensor 108, including for example a piezoelectric sensor. In one embodiment, pressure sensor 108 has a hole formed therein to receive one of the toilet seat mounting bolts. For example, pressure sensor 108 may be ring-shaped to provide the hole at or near its center.

In a sample embodiment, the pressure sensor comprises a first metal plate with a first wire attached thereto. The first metal plate is wrapped and sealed in a conductive material. The pressure sensor also comprises a second metal plate with similar dimensions as the first metal plate. The second metal plate is also wrapped and sealed in conductive material and also has a second wire attached thereto. The second wire is placed against the conductive material of the first metal plate. Both the first and second metal plates are then wrapped in nonconductive material. The pressure sensor is configured to emit an electronic signal when the two metal plates are compressed against each other. In a further embodiment, the resistance between the two plates drops when the plates are compressed against each other and the drop in resistance is detectable by the controller.

The filtration system 106 comprises a suction device 112, an odor sensor 114, and a filter 116. Suction device 112 is preferably disposed near the toilet to create negative pressure (sometimes also referred to as “suction”) near or inside the toilet bowl when the suction device is activated. When suction device 112 is activated, it directs air near or from inside the toilet bowl through the odor sensor 114 and the filter 116. The suction device 112 may direct air through the odor sensor 114 and filter 116 by either drawing or pushing air through the sensor 114 and filter 116. Arrows F in FIG. 1 show a possible direction of air flow when suction device 112 is activated.

In one embodiment, suction device 112 includes or is in communication with a conduit that is connectible to the toilet, for facilitating the suction of air from near or inside the toilet and/or for helping direct the air through the odor sensor and filter. The conduit may help provide a plenum, which is a space at positive or negative air pressure relative to the external pressure. One function of the plenum is to equalize air pressure for more even distribution of air therein, since the supply of or demand for air may vary from time to time. The plenum chamber may also function as an acoustic silencer to help reduce any noise generated by the suction device.

Suction device 112 may be any device that can generate air suction and draw or push air through the sensor 114 and filter 116. In one embodiment, suction device 112 is a fan, preferably an induction fan and/or high velocity fan. Induction fans operate on tunnel ventilation principles, producing a high velocity jet which adds momentum to the air in front of the fan, imparting thrust to all the surrounding air through mixing and entrainment as it diffuses. However, other types of fan may be used for suction device 112. In a further embodiment, suction device 112 is a fan that is DC-powered and is pulse width modulation (PWM) operated such that the power supplied thereto can be electronically controlled.

Odor sensor 114 is for sensing the presence and/or level of a particular compound(s) and/or pathogen(s) in the air passing therethrough. Certain compounds and pathogens can cause malodors. For example, H₂S is a foul smelling gas emitted during defecation. In one embodiment, odor sensor 114 is a type of sensor that senses the presence of H₂S. For instance, odor sensor may be a chemical membrane device that detects the presence of H₂S in the 0.25 ppm to 50 ppm range. Preferably, the odor sensor can detect any level of H₂S that is higher than 0.25 ppm. Other sensors that detect other offending compounds and/or pathogens may be used with system 100, depending on the application of system 100.

In a preferred embodiment, filter 116 comprises filter material that collects and removes at least some H₂S (and/or other offending compound(s) and/or pathogen(s) depending on the particular application) from the air passing therethrough. In a sample embodiment, the filter material is a mixture of carbon and potassium permanganate (KMnO₄). Of course, other filter materials may be used. After air containing offending compound(s) and/or pathogen(s) passes through the filter 116, the outgoing air should contain a smaller concentration of offending compounds and/or pathogen(s) and should be therefore less malodorous.

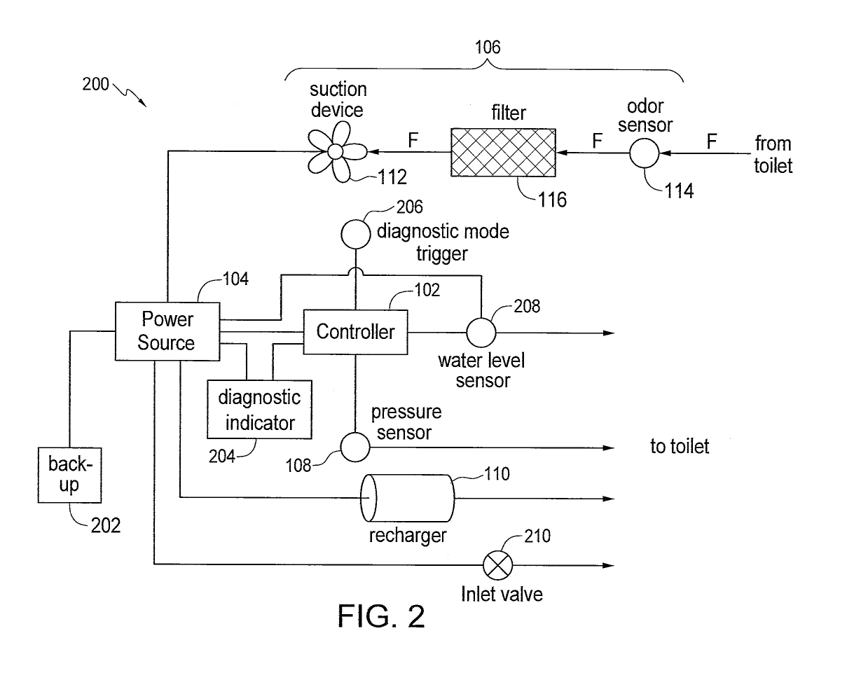

The system of the present invention may include additional components. Referring to FIG. 2, a system 200 is depicted, which comprises controller 102, power source 104, pressure sensor 108, recharger 110, and filtration system 106, all as described above with respect to system 100. Optionally, system 200 may further comprise one or more of: a backup power source 202; a diagnostic indicator 204; a diagnostic mode trigger 206; a water level sensor 208; and an inlet valve 210, each of which will be described in detail herein below.

The backup power source 202 provides backup power to one or more components of the system if power source 104 fails (e.g. malfunction, failure to recharge, lack of power supply from recharger 110, etc.). Backup power source 202 may be directly connected to each of the system components that may require power, or may be connected indirectly via power source 104. In one embodiment, the backup power source 202 comprises at least one battery. For example, the backup power source 202 may be three sets of two AA size alkaline batteries connected in series or three D-cell lithium batteries connected in series, to provide a backup voltage of about 9 VDC. Of course, other power sources that provide DC power may be used. The system 200 may include a power diode to (i) allow outflow of current from the backup power source 202 and/or (ii) block charge flow from recharger 110 to power source 104, if power source 104 fails.

The inlet valve 210 is for controlling the inflow of water into the toilet tank. More specifically, inlet valve 210 is used for shutting off, releasing, and/or distributing water to the toilet tank. Preferably, inlet valve 210 is an electromechanically operated valve that is controlled electronically. In a sample embodiment, valve 210 is a solenoid valve wherein the valve is opened or closed by sending an electric current through the solenoid. For example, the solenoid valve is open when the solenoid is energized by an electric current, thereby allowing fluid to flow therethrough; the solenoid valve is closed when the solenoid is not energized, thereby preventing fluid from flowing therethrough. In a preferred embodiment, inlet valve 210 is installable somewhere along the water inlet to the toilet tank in order to block water flow into the toilet tank when in the closed position, and to allow water flow into the toilet tank when in the open position.

The water level sensor 208 is for detecting the water level in the toilet tank and for emitting an electronic signal when the water in the toilet tank is at a certain level. In a sample embodiment, the water level sensor 208 comprises a first probe and a second probe. The first probe is in communication with controller 102 and the second probe is in communication with power source 104. One end of the first probe and one end of the second probe are placeable in the toilet tank, with the one end of the first probe at a lower vertical position than the one end of the second probe. For example, when the one end of the first probe is placed at a preselected “low” level within the toilet tank and the one end of the second probe is placed at a preselected “high” level, the water level in the toilet tank can be determined. More specifically, when the water level in the tank is at or above the preselected high level, the water is in contact with both probes at the one ends. Further, when the water level in the tank is below the preselected high level but above the preselected low level, the water is only in contact with the first probe. Still further, when the water level is below the preselected low level, the water is not in contact with either probe.

A possible way to determine the water level in the toilet tank relative to the preselected levels is to have the sensor send a signal (e.g. a voltage) to the controller 102 when the water in the tank is in contact with one or both probes. In a sample embodiment, when the water in the toilet tank is only in contact with the first probe, the first probe has a pull up voltage on the input of the controller 102, thereby signaling to the controller that the water level is at or above the preselected low level but below the preselected high level. When the water in the toilet tank is in contact with both probes, the sensor pulls down the voltage on the input of the controller 102, thereby signaling to the controller that the water in the tank is at or above the preselected high level. When the water in the tank is below the preselected low level, the water is not in contact with either probe and no signal is sent to the controller from the water level sensor. Of course, other sensor types and configurations, and other methods of detecting water level are possible.

In one embodiment, system 200 may include diagnostic mode trigger 206, which provides a possible way to initiate a diagnostic procedure in the system. The diagnostic mode trigger 206 is preferably in communication with controller 102. Various devices may be used for diagnostic trigger 206, including for example a sensor, a switch, etc. In a sample embodiment, diagnostic mode trigger 206 is a vibration sensor (e.g. a piezoelectric sensor), which is configured to detect vibration (or a specific pattern thereof) and to emit an electronic signal when vibration is detected. In a first illustrative example, when vibration is detected by the vibration sensor, it sends a signal to controller 102. Controller 102 may be preprogrammed to recognize and react to a particular signal pattern and/or frequency from the vibration sensor. For example, if controller 102 receives a certain number of signals from the vibration sensor within a predetermined time period (e.g. three signals within five seconds), controller 102 is triggered into diagnostic mode. In the diagnostic mode, the controller 102 checks the various components in the system.

In a second illustrative example, the vibration sensor is configured to detect a specific pattern of vibration (e.g. three consecutive vibrations) and to emit a signal to the controller 102 when the specific pattern of vibration is detected. When controller 102 receives a signal from the vibration sensor, the controller is triggered into diagnostic mode and proceeds to check the various components in the system.

In another sample embodiment, the diagnostic mode trigger 206 is a switch wherein the controller is triggered into diagnostic mode when the switch is switched from a first position to a second position.

In yet another sample embodiment, wherein diagnostic mode trigger 206 is omitted from the system, controller 102 may be configured to automatically enter into diagnostic mode periodically, such that it regularly checks the various components in the system.

The diagnostic indicator 204 provides visual indication of any problems and/or malfunctioning of the system. There are many ways to provide visual indication, including for example, an LED(s), a set of different coloured LEDs, a digital display, etc. As discussed above, when the controller 102 is triggered into diagnostic mode, it checks the various components in the system. If the controller determines that a particular component is malfunctioning, it signals the diagnostic indicator 204 to generate a visual indication. The visual indication may vary depending on which component is malfunctioning.

In a sample embodiment, the diagnostic indicator comprises an LED, which is not lit up unless signaled by the controller. If the controller is triggered into diagnostic mode and it finds that a component is malfunctioning, the controller sends a signal to the diagnostic indicator to light up the LED. In a further embodiment, if the malfunctioning component is, for example, the suction device, the controller may signal the diagnostic indicator to light up the LED constantly. In a still further embodiment, if the malfunctioning component is the power source, the controller may signal the diagnostic indicator to light up the LED periodically, for example at one second intervals. Of course, other visual indication variations are possible to indicate specific problems detected by the controller. Once the problem in the system is fixed, the controller sends a signal to the diagnostic indicator to reset to remove any visual indication.

In an alternative embodiment, power source 104 comprises an AC-to-DC power converter that is connectible to an AC power source (e.g. via wire and/or plug to an electrical wall outlet). The AC power source is preferably continuously readily available. In this alternative embodiment, the recharger is omitted. In this alternative embodiment, all system components, aside from the power source and the recharger, are the same as those described above with respect to FIGS. 1 and 2.

To operate system 100 or 200 with a toilet, pressure sensor 108 is installed at a location of the toilet where it can detect pressure when weight is put on the toilet seat (e.g. when a person sits on the toilet seat) and generate a signal. For example, pressure sensor 108 may be installed at one of the toilet seat bolts or under the toilet seat. As mentioned above, in a sample embodiment, pressure sensor 108 may include a hole, such that sensor 108 can be installed by placement between the toilet seat and the toilet bowl, with one of the mounting bolts passing through the hole in the sensor 108, and the toilet seat is secured to the toilet bowl in the usual manner.

The recharger 110, if included, is preferably installed somewhere in the flow path of the incoming water flow to the toilet water tank, such that recharger 110 is driven by the flow of incoming water as the tank is filled.

Suction device 112 is preferably situated at or near the toilet to allow it to create negative pressure inside or near the toilet bowl. If the system includes the conduit for facilitating the suction of air from near or inside the toilet, one end of the conduit is installed at a desired location near or inside the toilet, and the other end of the conduit is in communication with suction device 112.

If water level sensor 208 is included in the system, the water level sensor or at least a part thereof is installed at a location where it can detect the water level in the toilet tank; for example, a part of the water level sensor may be installed inside the toilet tank.

If the inlet valve 210 is included in the system, valve 210 is preferably installed somewhere along the water supply inlet to the toilet tank such that it can control the flow of water into the toilet tank.

If the system includes diagnostic mode trigger 206 and diagnostic indicator 204, the diagnostic indicator is preferably installed at a location that is visible to a user.

A method according to the present invention for deodorizing air at or near a toilet comprising a toilet seat, a toilet tank, and a toilet bowl, the method comprises: detecting pressure on the toilet seat; directing air from near or inside the toilet bowl through a filter at a first rate if pressure on the toilet seat is detected; detecting presence of H₂S; directing air from near or inside the toilet bowl through the filter at a second rate if H₂S is detected, the second rate being higher than the first rate; detecting removal of pressure from the toilet seat; and ceasing the direction of air through the filter after a predetermined period of time if no H₂S is detected or if removal of pressure on the toilet seat is detected.

The method may further comprise converting hydraulic power from an inflow of water into the toilet tank into electrical power; and recharging a power source with the electrical power. The method may further comprise supplying power from a backup power source if there is a lack of power in the power source.

The method may further comprise selectively controlling water flow into the toilet water tank and/or detecting the water level in the toilet water tank. The method may comprise detecting the water level in the toilet tank and selectively controlling water flow to the toilet tank with respect to the detected water level. In a further embodiment, the method comprises performing a diagnostic check on at least one system component; and providing a visual indication if the at least one system component is malfunctioning. The method may further comprise receiving a signal to trigger the performance of the diagnostic check.

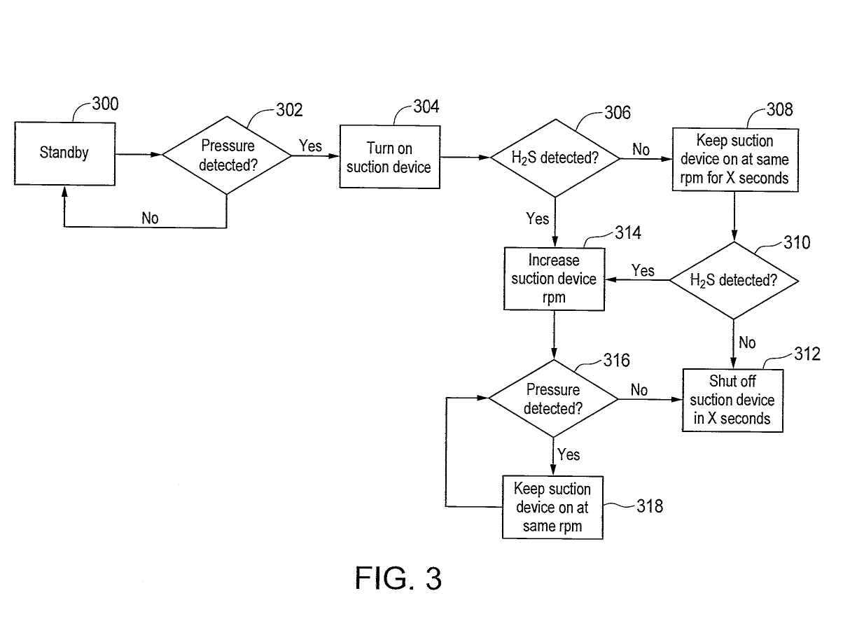

FIG. 3 illustrates a sample method operable with system 100, 200. In operation, when the toilet is empty (i.e. no one on the toilet seat), the system is in standby mode (step 300), wherein the pressure sensor is active and ready to detect pressure while all other system components are idle. As long as the pressure sensor does not detect any pressure (step 302), the system stays in standby mode (step 300). When the pressure sensor senses pressure (i.e. when a person sits on the toilet seat), the pressure sensor sends a signal to the controller. After receiving the signal from the pressure sensor, the controller signals the power source to supply power to the suction device, allowing it to operate to begin sucking air from near or inside the toilet bowl (step 304). In a preferred embodiment, a low power supply is fed to the suction device to keep it operating at a low suction rate at this initial stage. The suction device creates negative pressure near or inside the toilet bowl to direct air therefrom across the odor sensor and through the filter. In a preferred embodiment, as air passes through the odor sensor, the odor sensor detects the presence of H₂S in the air (step 306).

The system may be configured to allow the suction device to operate for a predetermined amount of time, which may or may not depend on the presence of weight on the toilet seat and/or the presence or lack of H₂S detected by the odor sensor.

In a sample embodiment, if the odor sensor does not detect any H₂S, the system keeps the suction device running for a predetermined time period, for example 20 seconds, (step 308) and, optionally, the sensor keeps checking for H₂S in the meantime (step 310). If no H₂S is detected at the end of the predetermined time period, the controller sends a signal to the power source to stop supplying power to the suction device after a predetermined amount of time (e.g. 15 seconds), thereby shutting off the suction device (step 312).

If H₂S is detected (steps 306, 310), the odor sensor sends a signal to the controller, which in turn signals the power source to increase its power supply to the suction device, thereby increasing the rate at which air is drawn from near or inside the toilet bowl and at which air flows through the filter (step 314). If the pressure sensor still detects pressure on the toilet seat (step 316), the system keeps the suction device on at the increased suction rate (step 318). If the pressure sensor does not detect any pressure on the toilet seat, the system shuts off the suction device after a predetermined amount of time (step 312).

When the toilet is flushed, the toilet tank is emptied and water is supplied anew to the toilet tank though the water supply inlet. If the recharger is included, as the incoming water from the inlet flow through the recharger, the recharger delivers power to the power source to recharge same.

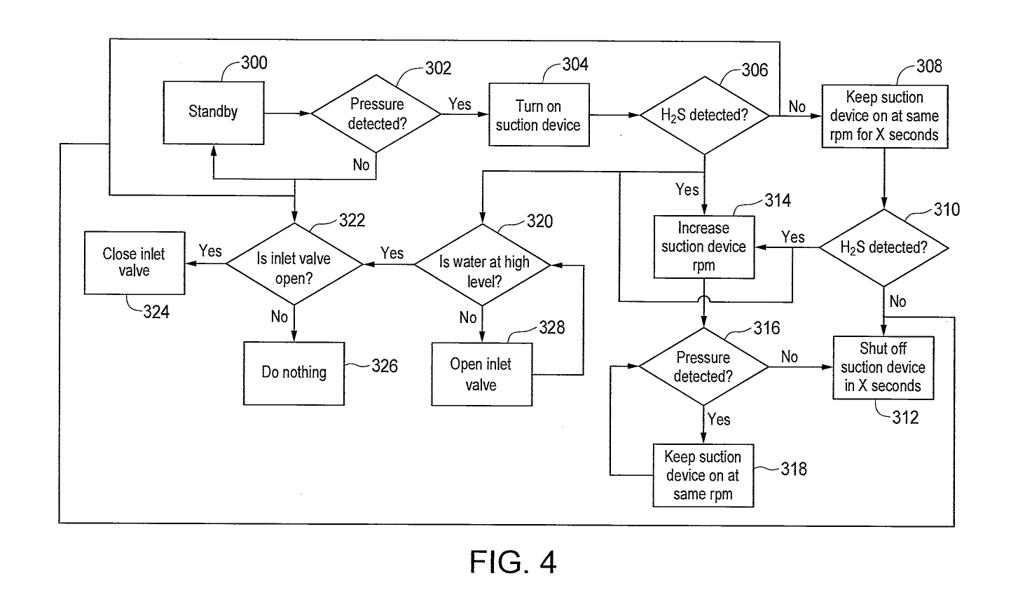

In a further embodiment, the system may include the water level sensor and inlet valve to help control water usage of the toilet. FIG. 4 illustrates a sample method operable with system 200 comprising the water level sensor and inlet valve. In this embodiment, each time the toilet is flushed, the toilet tank is refilled to only the preselected low level. For example, when the toilet is flushed and the toilet tank is emptied, the controller signals the power source to open the inlet valve, whether by supply power to it or ceasing the supply of power to it, depending on the configuration of the inlet valve. When the inlet valve is in the open position, water can flow into the toilet tank to increase the water level inside the toilet tank. In a preferred embodiment, when the water level senses that the water level inside the tank has reached the preselected low level, it sends a signal to the controller, which in turn signals the power source to close the valve.

If the pressure sensor senses pressure on the toilet seat (step 302) and the odor sensor detects the presence of H₂S (step 306), the system checks the water level in the toilet tank using the water level sensor (step 320). If the water level sensor senses that the water level in the toilet tank is at or above the preselected high level (step 320), the system checks whether the inlet valve is open (step 322). If the inlet valve is open (step 322), the controller sends a signal to the power source and the power source either supplies power or ceases the supply of power to the inlet valve to close the valve (step 324). If the inlet valve is already closed (step 322), the system does not react (step 326). If the water level sensor senses that the water level in the toilet tank is below the preselected high level (step 320), the sensor sends a signal to the controller, which in turn signals the power source to either supply power or cease the supply of power to the inlet valve to open the valve (step 328), to allow water to enter the toilet tank. The inlet valve remains open until the water level sensor senses that the water level in the toilet tank has reached the preselected high level (step 320). In one embodiment, the system may leave the inlet valve open until the water level in the tank reaches the preselected high level, whether or not the pressure sensor senses pressure on the toilet seat in the meantime.

If the pressure sensor does not detect any pressure on the toilet seat (step 302) and/or if the odor sensor does not detect any H₂S (step 306, 310), the system operates to maintain the current water level (e.g. preselected low level) in the toilet tank by first checking the inlet valve to see whether it is open (step 322). If the inlet valve is open (step 322), the controller signals the power source to close the inlet valve (step 324). If the inlet valve is already closed (step 322) the system does not react (step 326). Preferably, when the toilet tank is filled, the water level in the tank is kept at the preselected low level unless H₂S is detected.

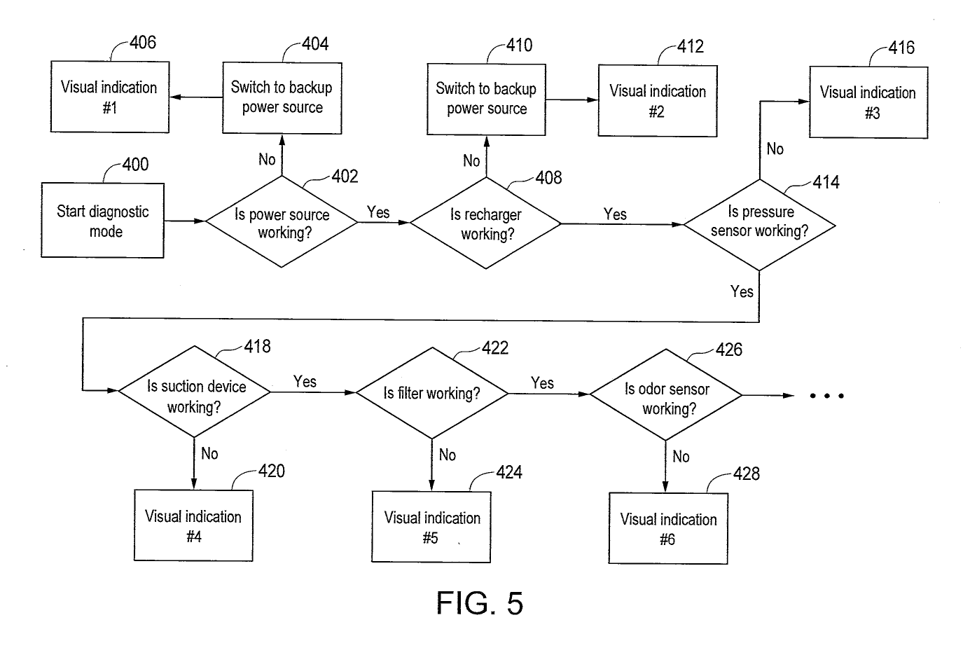

In a still further embodiment, the system may include the diagnostic indicator and optionally the diagnostic mode trigger. FIG. 5 illustrates a sample method operable with system 200 comprising the diagnostic indicator and diagnostic capabilities. The system starts the diagnostic mode (step 400), which may be triggered by the diagnostic mode trigger, if it is included in the system, or may be initiated automatically by the controller. The controller checks the various components in the system.

The controller checks the power source (step 402). If the power source is malfunctioning, the power supply for the system is switched to the backup power source, if included (step 404) and the controller signals the diagnostic indicator to display a first visual indication (step 406). If the power source is working properly, the controller checks the recharger, if included (step 408), for example by measuring the output voltage generated by the recharger. If the recharger is malfunctioning, the power supply for the system is switched to the backup power source (step 410) and the controller signals the diagnostic indicator to display a second visual indication (step 412). If the recharger is functioning normally, the controller checks the pressure sensor (step 414). If the pressure sensor is malfunctioning, the controller signals the diagnostic indicator to display a third visual indication (step 416). If the pressure sensor is working properly, the controller checks the suction device (step 418). If the suction device is malfunctioning, the controller signals the diagnostic indicator to display a fourth visual indication (step 420). If the suction device is functioning normally, the controller checks the filter (step 422). If the filter is malfunctioning, the controller signals the diagnostic indicator to display a fifth visual indication (step 424). If the filter is working properly, the controller checks the odor sensor (step 426). If the odor sensor is malfunctioning, the controller signals the diagnostic indicator to display a sixth visual indication (step 428). If the odor sensor is functioning normally, the controller may continue to check other components in the system, including for example, the water level sensor and the inlet valve.

The method depicted in FIG. 5 shows one sample sequence under the diagnostic mode. Of course, other sequences are possible and two or more of steps 402, 408, 414, 418, 422, and 426 may take place simultaneously. Further, each visual indication may be different from the other visual indications or may be the same as one or more of the other visual indications.

To embody the above-described system and/or to implement the above-described method, a device for deodorizing air at or near a toilet comprising a toilet seat, a toilet tank, and a toilet bowl is described herein. In one embodiment, the device comprises: a outer housing having a perforated first end and a perforated second end; a filter medium disposed inside and between the first and second ends of the outer housing; a suction device disposed inside the outer housing, the suction device configured to selectively draw air through the filter medium; an odor sensor disposed on or inside the outer housing, the odor sensor being in communication with the controller; a pressure sensor in communication with the controller, the pressure sensor for detecting changes in pressure on the toilet seat; a controller disposed inside the outer housing for receiving signals from the pressure sensor and the odor sensor, and for selectively operating the suction device; a power source electrically connected to the suction device, the controller, the odor sensor and the pressure sensor, for supplying power to same; and a conduit assembly connected to the first end of the outer housing, for facilitating the suction of air from near or inside the toilet by the suction device and/or for directing the air through the odor sensor and the filter medium.

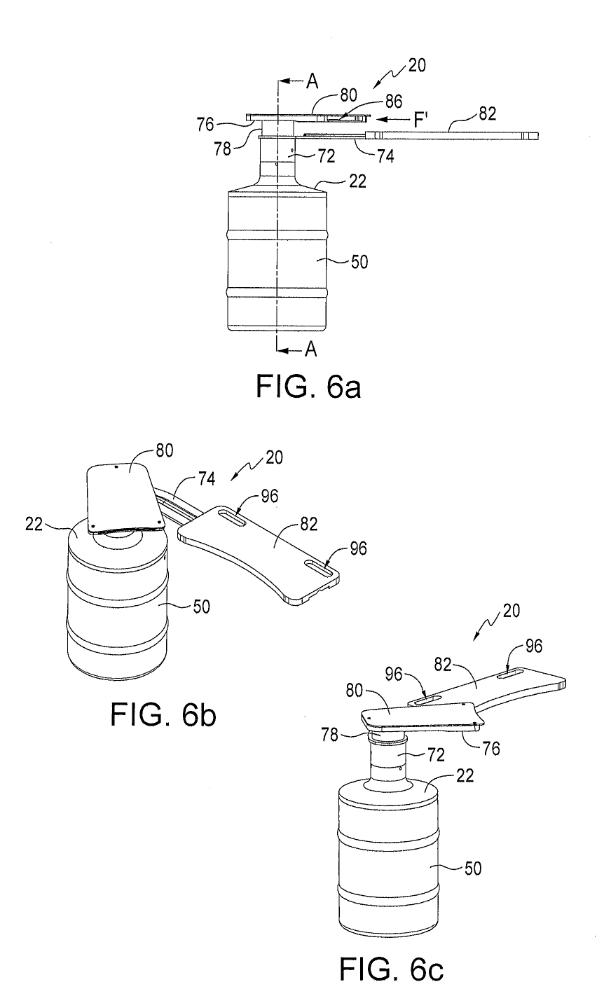

FIGS. 6 to 8 depict a sample device 20 embodying the system of the present invention for carrying out the above-described methods.

Device 20 comprises a suction device 36, a filter, an odor sensor 23, a power source 49, a controller (not shown), and a pressure sensor. Device 20 further comprises a conduit connectible to the toilet for facilitating air suction by suction device 36 from near or inside the toilet.

In the illustrated embodiment, suction device 36 is a fan that when activated, creates negative pressure in the space adjacent to a first side 37 thereof. Preferably, the fan is DC powered and pulse width modulation (PWM) operated.

The power source is supported by a power source housing 34. In one embodiment, the power source is one or more ultra-capacitors. The power source housing 34 may optionally be configured to further support a backup power source 49. In the illustrated embodiment, the power source housing 34 has a generally cylindrical shape with a first open end 44, a second open end 46, and an axial bore 42 extending therethrough. Power source housing 34 has compartments 48 on its outer surface for receiving and supporting optional backup power source 49, which may be for example one or more batteries.

In an alternative embodiment, instead of ultra-capacitors, the power source is a converter (not shown) for converting the AC power to DC power, and the converter is connectible to an AC power source, for example by wire and/or plug to an electrical wall outlet. In this alternative embodiment, the power source housing is configured to accommodate the converter.

In the illustrated embodiment of device 20, the first side 37 of suction device 36 is disposed adjacent to the second end 46 of power source housing 34 and is in fluid communication with bore 42. While suction device 36 is shown as being outside housing 34, suction device 36 may be disposed inside housing 34, for example inside bore 42.

The filter of device 20 is a filter body 32 having filter medium 35 therein. In a sample embodiment, the filter body 32 is a tubular member having an open first end 38, a second end 39, and an axially-extending bore 41 extending between ends 38 and 39. Filter body 32 may further comprise a filter cap 40 for covering first end 38. Both second end 39 and filter cap 40 are perforated to allow passage of air therethrough. In this description, the term “perforated” refers to a surface that air-permeable, including a surface that has an opening, that is perforated, porous, etc.

Filter cap 40 may be fit on first end 38 in many ways. For example, filter cap 40 may be shaped to mate with first end 38. In the illustrated embodiment, filter cap 40 has an axially-extending ridge along its circumference and first end 38 includes a circumferential ledge for receiving and supporting the ridge of filter cap 40. Of course, other ways of connecting filter cap 40 and first end 38 are possible, including for example, peg and slot interface, threaded connections, friction fitting, etc. In another embodiment, filter cap 40 is not directly connected to the filter body, but is instead held in place relative to the filter body by other components of the device.

In one embodiment, the filter medium comprises layers of filter material and fills up substantially all of the interior space of filter body 32. The filter medium is selected to remove H₂S and pathogens in the air passing therethrough and includes for example a mixture of carbon and potassium permanganate (KMnO₄).

Bore 42 is sized to receive at least a portion of filter body 32. In the illustrated embodiment, filter body 32 is disposed in bore 42 (with second end 39 in the direction of second end 46), with the long central axis of bore 41 is substantially aligned with the long central axis of bore 42, and first end 38 of filter body 32 is at or near first end 44 inside bore 42 of power source housing 34.

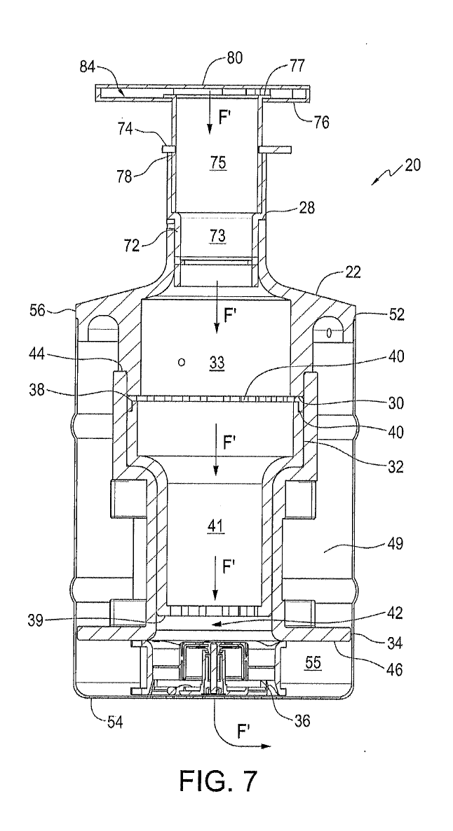

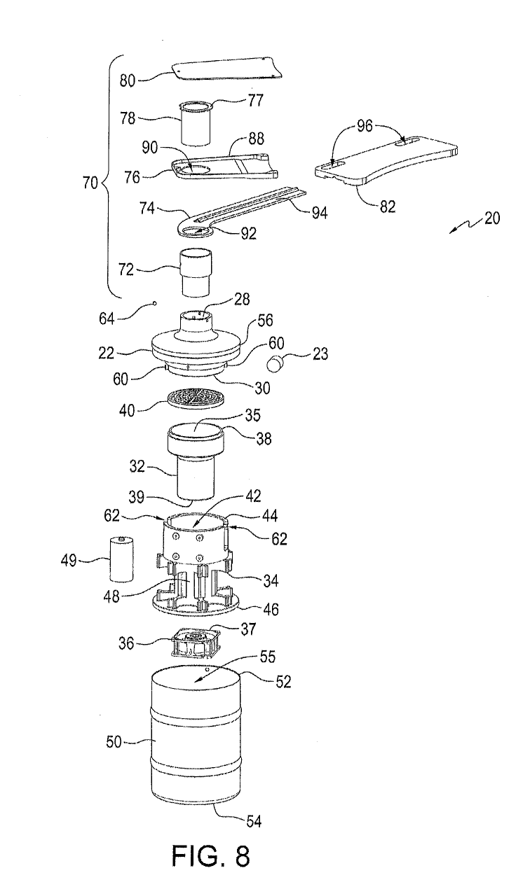

Device 20 further comprises a cap 22 for housing therein the odor sensor. Cap 22 has a first open end 28 and a second open end 30, and an axially extending inner bore 33 between ends 28 and 30. Preferably, cap 22 is disposed adjacent to housing 34 and filter body 32, with second end 30 facing ends 38 and 44, and bore 33 in communication with bores 41 and 42 via filter cap 40. In one embodiment, second end 30 of cap 22 is configured to connect and mate with end 44. For example, as shown in FIGS. 7 and 8, cap 22 has at least one pin 60 on its outer surface near second end 30 and housing 34 has at least one slot 62 at end 44, each for receiving a pin 60. This way, cap 22 may interlock with housing 34 by inserting the at least one pin 60 into the at least one slot 62. Of course, other ways of connecting cap 22 and housing 34 are possible, including for example, threaded connections, friction fitting, etc. In one embodiment, filter cap 40 is instead held in place relative to the filter body by the connection between cap 22 and housing 34.

Alternatively or additionally, end 30 of cap 22 and end 44 of housing 34 may be shaped to mate with each other. For example, in the illustrated embodiment, end 30 is shaped to provide a shoulder for receiving end 44. In one embodiment, when end 44 is received in the shoulder at end 30, end 30 abuts end 38 of filter body 32 (or filter cap 40, if it is included), thereby helping to keep filter body 32 inside bore 42. In one embodiment, filter cap 40 may be shaped to mate with first end 38 and/or second end 30 to fit securely therebetween.

Device 20 may further comprise an outer housing 50 to provide covering and protection for filter body 32, power source housing 34, and suction device 36. Outer housing 50 may also help keep the components in place relative to one another. In one embodiment, outer housing 50 has a generally cylindrical shape with an open first end 52, and a second end 54, and an axial bore 55 extending therethrough. The axial bore of outer housing 50 is sized and shaped to accommodate therein suction device 36, power source housing 34, with backup power source 49 supported thereon and filter body 32 disposed therein, and at least a portion of cap 22. In a further embodiment, suction device 36 is disposed adjacent to end 54 and cap 22 adjacent to end 52, with power source housing 34 and filter body 32 therebetween. At least a portion of second end 54 is perforated to allow passage of air therethrough to suction device 36.

Cap 22 is configured to be securable to end 52 of housing 50. For example, as shown FIGS. 7 and 8, cap 22 has a radially outwardly extending flange 56, which has a reduced diameter portion for receiving end 52 of outer housing 50. Fasteners, such as screws, may be used to secure cap 22 to the outer housing at the reduced diameter portion. Of course, other ways of connecting cap 22 and housing 50 are possible, including for example, a peg and slot interface, threaded connections, friction fitting, etc.

In a further alternative embodiment, especially where the power source is a converter connectible to an AC power source and/or where the backup power source is omitted, the power source housing may be omitted, such that the outer housing accommodates the power source directly therein. Further, in this embodiment, filter cap 40 may be held in place relative to the filter medium and/or filter body by the connection between cap 22 and housing 50. Preferably, filter cap 40 is disposed within outer housing 50 near end 52 thereof.

In a still further alternative embodiment, filter body 32 is omitted and the filter medium is housed directly within the outer housing 50, for example, between suction device 36 and filter cap 40.

Suction device 36, the controller, the odor sensor and the pressure sensor are electrically connected (e.g. by wires), directly or indirectly, to the power source and the backup power source, if included, such that they can receive electrical power therefrom. The recharger, if included, is electrically connected to the power source such that it can provide power thereto. If the diagnostic indicator, the inlet valve, and/or the water level sensor are included, each of these components is electrically connected, directly or indirectly, to the power source and the backup power source, if included, such that they can receive electrical power therefrom.

The recharger and the inlet valve are electrically connected to the power source, for example, via a keyed cable connector.

In a sample embodiment, the diagnostic indicator is disposed on the outer surface of cap 22 such that it is visible to a user.

Device 20 may further comprise a conduit assembly 70 for connecting device 20 to the toilet and for facilitating the suction of air from near or inside the toilet by the suction device and/or for helping direct the air through the odor sensor and filter. In a sample embodiment, the conduit assembly 70 comprises a coupler 72, a slider 74, a lower top 76, a top post 78, an upper top 80, and a base 82.

Coupler 72 and top post 78 are tubular members each having an open first end and an open second end and an axially-extending inner bore therebetween. Coupler 72 connects the conduit assembly 70 with the other parts of device 20. In one embodiment, coupler 72 is connected to cap 22 such that a portion of coupler 72 extends inside bore 33 of cap 22. In a further embodiment, coupler 72 has a circumferential shoulder on its outer surface in which first end 28 is received, as shown in FIG. 7. The inner bore 73 of coupler 72 is in fluid communication with inner bore 33 of cap 22, which in turn is in communication with bores 41 and 42.

Coupler 72 and top post 78 are configured to connect with one another. In one embodiment, a portion of top post 78 extends into bore 73 of coupler 72. For example, as shown in FIG. 7, coupler 72 has an inner circumferential shoulder on the surface of its inner bore in which the second end of top post 78 is received. Preferably, the central axial axis of inner bore 75 of top post 78 is substantially aligned with that of inner bore 73 of coupler 72. Inner bore 75 is in fluid communication with inner bore 73, which in turn is in communication with inner bores 33, 41 and 42. The first end of top post 78 has a radially outwardly extending flange 77.

In a preferred embodiment, conduit assembly 70 provides a thin air channel with a small narrow inlet for placement near or inside the toilet bowl for drawing air therethrough towards the filter. There are many ways the thin air channel and small narrow inlet may be provided. For example, in the illustrated embodiment, lower top 76 and upper top 80 are plate-like members having substantially planar surfaces that when combined provide a thin air channel 84 therebetween with a small narrow inlet 86.

More specifically, lower top 76 has a ridge 88 at a substantial portion of its outer edge, and a hole 90 for receiving top post 78 therethrough. The outer diameter of flange 77 is larger than the diameter of hole 90 such that all but the flange of top post 78 can fit through the hole. The lower surface of flange 77 abuts the rim of hole 90 and may be held in place by adhesives, fasteners, friction fitting, or a combination thereof. Upper top 80 is secured to the lower top 76 at the top edge of the ridge 88, such that a planar surface of upper top 80 is facing and is substantially parallel to a planar surface of lower top 76, and the planar surfaces are separated by a distance (i.e. the height of ridge 88). The space between lower top 76 and upper top 80 forms air channel 84. The portion of lower top 76 that does not have a ridge forms inlet 86, through which air can enter air channel 84. In one embodiment, one of the planar surfaces of lower top 76 and upper top 80 may include a step or a ramp, such that the height of the air channel is increased or decreased across the step or ramp. The air channel 84 is in fluid communication with inner bores 75, 73, 33, 41 and 42.

The components of the device 20 may be separate components, or one or more components may be manufactured together as a single component. For example, in one embodiment, top post 78 and lower top 76 are one piece of material. In a further example, coupler 72 and cap 22 are a single piece of material. Other parts may be also be combined such that the device comprises fewer separate components.

There are many possible ways to connect device 20 to the toilet. Preferably, device 20 connects to the toilet at the toilet seat mounting bolts under the toilet seat. In the illustrated embodiment, conduit assembly 70 includes slider 74 and base 82 for connecting device 20 to the toilet. More specifically, slider 74 has a hole 92 at a first end for receiving top post 78 therethrough and an arm 94 extending away from the hole for mating connection with base 82. In the illustrated embodiment, the diameter of hole 92 is substantially the same as the inner diameter of the first end of coupler 72 and the outer diameter of top post 78, such that top post 78 fits through the hole 92 and extends into bore 73 of coupler 72, with a portion of slider 74 around hole 92 resting on the first end of coupler 72. A fastener 64 may be used to secure slider 74 to top post 78. The fastener 64 may be, for example, a set screw.

In the illustrated embodiment, base 82 is a plate-like member having at least two holes 96, each for receiving a toilet seat mounting bolt therethrough. Base 82 also includes a track, which is configured to matingly receive at least a portion of arm 94 of slider 74, such that arm 94 is slidable into and out of base 82. The pressure sensor is installed in or under base 82, preferably at a location where it can easily detect pressure on the toilet seat when base 82 is connected to the toilet at the toilet seat bolts. In one embodiment, the pressure sensor is installed near one of holes 96. In another embodiment, the pressure sensor is connected to base 82 such that when pressure is placed on the toilet seat, the base 82 flexes and compresses the pressure sensor.

Any two adjacent parts mentioned above (e.g. suction device 36, power source housing 34, filter body 32, cap 22, coupler 72, etc.) may be connected by friction-fitting, threaded connection, fasteners, adhesives, welding, bonding, or a combination thereof. Inside housing 50, the parts may or may not be secured to one another and may simply be held in place by the configuration and sizing of housing 50 and/or each of the parts. For example, the length of bore 55 may be selected such that when suction device 36, power source housing 34, and a portion of cap 22 are placed inside housing 50, with ends 37 and 46, and ends 44 and 30 abutting, and cap 22 secured to end 52, the parts are in frictional contact with one another and/or with the inner surface of housing 50.

In one embodiment, top post 78 is rotatable about its central axial axis when it is inserted inside coupler 72, such that the position of the inlet 86 relative to the slider 74 and the base 82 is selectively adjustable. Alternatively or additionally, lower top 76 and upper top 80 are pivotable at hole 90 about the central axial axis of the top post. In a further embodiment, slider 74 is pivotable at hole 92 about the central axial axis of coupler 72, such that the location of lower top 76 and upper top 80 relative to base 82 is selectively adjustable. The distance between inlet 86 (and other parts of device 20) and base 82 may also be selectively adjusted by sliding arm 94 into and/or out of base 82.

Cap 22, filter body 32, filter cap 40, power source housing 34, conduit assembly 70, and outer housing 50 may be made of plastic, such as PVC, or any other material suitable for the device's intended use. In the event that any of the device's components is intended to be disposable, it desirably may be made of a biodegradable material, and can even be made of wood or other cellulosic material.

To use device 20 with a standard toilet, base 82 is installed between the toilet seat and the toilet bowl, by inserting and securing the toilet seat mounting bolts through holes 96. Arm 94 of slider 74 is inserted into the track of base 82. The distance of inlet 86 (and other parts of device 20) from the toilet can be selectively adjusted by sliding arm 94 into and/or out of base 82, and/or by pivoting upper top 80 and lower top 76. Preferably, inlet 86 is positioned to be near and/or to face the toilet bowl.

In operation, when pressure is exerted on the toilet seat (e.g. when a person sits on the toilet seat), the pressure sensor detects the pressure and sends a signal to the controller to activate the suction device 36. When suction device 36 is activated, suction device 36 generates negative air pressure at side 37, thereby drawing air from inlet 86 through air channel 84, inner bores 75, 73, 33, and filter body 32, respectively. The direction of air flow when suction device 36 is activated is denoted by arrows F′. The odor sensor 23 detects the presence H₂S in the air that passes therethrough. If H₂S is detected, the odor sensor sends a signal to the controller and the controller reacts as described above. The filter material in filter body 32 helps filter out any H₂S and other pathogens in the air passing therethrough. The air exiting the filter body 32 is drawn through the suction device 36 and emitted from outer housing 50 at the perforated portion of end 54.

In a sample embodiment, device 20 includes a vibration sensor. Device 20 is triggered into starting the diagnostic mode when the vibration sensor senses a preselected frequency of vibrations (e.g. three physical taps on the outer housing 50 within two seconds). In the diagnostic mode, device 20 operates as described above.

The previous description of the disclosed embodiments is provided to enable any person skilled in the art to make or use the present invention. Various modifications to those embodiments will be readily apparent to those skilled in the art, and the generic principles defined herein may be applied to other embodiments without departing from the spirit or scope of the invention. Thus, the present invention is not intended to be limited to the embodiments shown herein, but is to be accorded the full scope consistent with the claims, wherein reference to an element in the singular, such as by use of the article “a” or “an” is not intended to mean “one and only one” unless specifically so stated, but rather “one or more”. All structural and functional equivalents to the elements of the various embodiments described throughout the disclosure that are know or later come to be known to those of ordinary skill in the art are intended to be encompassed by the elements of the claims. Moreover, nothing disclosed herein is intended to be dedicated to the public regardless of whether such disclosure is explicitly recited in the claims. For US patent properties, it is noted that no claim element is to be construed under the provisions of 35 USC 112, sixth paragraph, unless the element is expressly recited using the phrase “means for” or “step for”.

I claim:

1. A method for deodorizing air at or near a toilet comprising a toilet seat, a toilet tank, and a toilet bowl, the method comprises:
detecting pressure on the toilet seat;
directing air from near or inside the toilet bowl through a filter at a first rate if pressure on the toilet seat is detected;
detecting presence of H₂S;
directing air from near or inside the toilet bowl through the filter at a second rate if H₂S is detected, the second rate being higher than the first rate;
detecting removal of pressure from the toilet seat; and
ceasing the direction of air through the filter after a predetermined period of time if no H₂S is detected or if removal of pressure on the toilet seat is detected.

2. The method of claim 1 further comprising converting hydraulic power from an inflow of water into the toilet tank into electrical power; and
recharging a power source with the electrical power.

3. The method of claim 2 further comprising supplying power from a backup power source if there is a lack of power in the power source.

4. The method of claim 1 further comprising selectively controlling water flow to the toilet tank.

5. The method of claim 1 further comprising detecting the water level in the toilet tank and selectively controlling water flow to the toilet tank with respect to the detected water level.

6. The method of claim 1 wherein detecting pressure on the toilet seat is performed by a pressure sensor in communication with a controller; directing air from near or inside the toilet bowl is performed by an air filtration system in communication with the controller; and detecting the presence of H₂S is performed by an odor sensor of the air filtration system, and the method further comprising performing a diagnostic check on at least one of: the pressure sensor, the controller, the air filtration system, and the odor sensor.

7. The method of claim 6 further comprising providing a visual indication if any one of the pressure sensor, the controller, the air filtration system, and the odor sensor is malfunctioning.

8. The method of claim 6 further comprising receiving a signal to trigger the performance of the diagnostic check.

9. The method of claim 6 wherein a power source supplies power to one or more of the pressure sensor, the controller, the air filtration system, and the odor sensor, and the method further comprising performing a diagnostic check on the power source and, if the power source is malfunctioning, supplying power to one or more of the pressure sensor, the controller, the air filtration system, and the odor sensor using a backup power source.

10. The method of claim 2 wherein converting hydraulic power into electrical power is performed by a recharger and the further comprising: performing a diagnostic check on the recharger; and, if the recharger is malfunctioning, supplying power using a backup power source instead of the power source.

11. A deodorizing system for operation with a toilet having a toilet seat and a water tank, the system comprising:
a controller;
a pressure sensor for sensing pressure on the toilet seat and for communicating same to the controller via a first signal;
an air filtration system for filtering air near the toilet upon receiving the first signal from the controller; and
a power source for supplying power to the controller and the air filtration system.

12. The deodorizing system of claim 11 further comprising a recharger installable in an inlet to the water tank, the recharger for converting hydraulic power to electrical power and for supplying electrical power to the power source.

13. The deodorizing system of claim 11 wherein the power source comprises ultra-capacitors.

14. The deodorizing system of claim 12 wherein the recharger is a micro-turbine.

15. The deodorizing system of claim 11 wherein the pressure sensor is a piezoelectric sensor.

16. The deodorizing system of claim 11 wherein the filtration system comprises:
a suction device for selectively creating negative pressure near or inside the toilet;
an odor sensor for detecting a level of at least one odor causing compound and/or pathogen above a threshold and for sending a second signal to the controller upon detecting same; and
a filter for collecting and/or removing at least some of the odor causing compound and/or pathogen, and
wherein the suction device creates negative pressure near or inside the toilet upon receiving a second signal from the controller and directs air through the odor sensor and the filter.

17. The deodorizing system of claim 16 wherein the odor causing compound and/or pathogen is H₂S and the threshold ranges from about 0.25 ppm to about 50 ppm.

18. The deodorizing system of claim 16 wherein the filter comprises carbon and potassium permanganate.

19. The deodorizing system of claim 16 wherein the suction device is in communication with a conduit connectible to the toilet, the conduit for one or both of: (i) facilitating the suction of air from near or inside the toilet; and (ii) directing air through the odor sensor and filter.

20. The deodorizing system of claim 16 wherein the suction device is a fan.

21. The deodorizing system of claim 11 further comprising one or more of:
a backup power source;
a diagnostic indicator for indicating any problems and/or malfunctioning of the system;
a diagnostic mode trigger in communication with the controller;
a water level sensor for detecting the water level in the water tank and for emitting an electronic signal to the controller with respect to same; and
an inlet valve for selectively shutting off, releasing, and/or distributing water into the water tank.

22. The deodorizing system of claim 21 wherein the inlet valve is an electronically controlled solenoid valve.

23. The deodorizing system of claim 21 wherein the diagnostic mode trigger is a vibration sensor.

24. The deodorizing system of claim 21 wherein the diagnostic indicator provides visual indication.

25. A device for deodorizing air at or near a toilet comprising a toilet seat, a toilet tank, and a toilet bowl, the device comprises:
a outer housing having a perforated first end and a perforated second end;
a filter medium disposed inside and between the first and second ends of the outer housing;
a suction device disposed inside the outer housing, the suction device configured to draw air through the filter medium when in operation;
an odor sensor disposed on or inside the outer housing, the odor sensor being in communication with the controller;
a pressure sensor in communication with the controller, the pressure sensor for detecting changes in pressure on the toilet seat;
a controller disposed inside the outer housing for receiving signals from the pressure sensor and the odor sensor, and for selectively operating the suction device;
a power source electrically connected to the suction device, the controller, the odor sensor and the pressure sensor, for supplying power to same; and
a conduit assembly connected to the first end of the outer housing, for facilitating the suction of air from near or inside the toilet by the suction device and/or for directing the air through the odor sensor and the filter medium.

26. The device of claim 25 wherein the filter medium comprises a mixture of carbon and potassium permanganate.

27. The device of claim 25 wherein the conduit assembly comprises:
a coupler having an inner bore, the coupler is connected to the first end of the outer housing, the inner bore being in fluid communication with the filter medium and the suction device;
a lower top having an inner surface and a hole therein; and
an upper top having an inner surface, the upper top connected to the lower top such that the inner surfaces of the lower top and the upper top define a thin air channel therebetween, the thin air channel having a small narrow inlet, the thin air channel being in fluid communication with the inner bore of the coupler via the hole, and inlet being positionable inside or near the toilet bowl.

28. The device of claim 27 wherein the conduit assembly further comprising:
a slider having a first end connected to the coupler and an arm extending from the first end; and
a base configured to slidably connect with the arm and to be mountable to the toilet by at least one toilet seat mounting bolt of the toilet.

29. The device of claim 28 wherein the pressure sensor is installed to the base.

30. The device of claim 27 wherein the lower top and upper top are pivotable at the hole about a central axial axis of the coupler.

31. The device of claim 28 wherein the arm of the slider is pivotable about a central axial axis of the coupler.

32. The device of claim 25 further comprising a cap connecting the first end of the outer housing to the conduit assembly, the cap having an open first end and an open second end and a bore extending therebetween, the bore for providing an air passage between the conduit assembly and the filter medium.

33. The device of claim 25 wherein the power source is connectable to a recharger installed in a water inlet to the toilet tank, the recharger for converting hydraulic power to electrical power and for supplying electrical power to the power source.

34. The device of claim 25 further comprising a backup power source electrically connected to the suction device, the controller, the odor sensor and the pressure sensor, for supplying power to same.

35. The device of claim 25 wherein the controller and the power source are in communication with an inlet valve installed in an inlet to the toilet tank, and the inlet valve is controllable by the controller to selectively allow and/or restrict water flow to the toilet tank.

36. The device of claim 25 wherein the controller and the power source are in communication with a water level sensor installed in the toilet tank, and the water level sensor is configured to detect a water level in the toilet tank and to send a signal to the controller with respect to same.

37. The device of claim 25 further comprising a diagnostic indicator in communication with the controller and the power source.

38. The device of claim 37 wherein the diagnostic indicator is visible on the outer housing.

39. The device of claim 25 wherein the filter medium is housed in a filter body with a perforated first end, a perforated second end, the filter body being disposed inside the outer housing with its second end near the second end of the outer housing.

40. The device of claim 39 wherein the first end of the filter body includes a perforated filter cap.

41. The device of claim 25 wherein at least part of the device is made of a biodegradable material.
